(19) United States
(12) Patent Application Publication
(10) Pub. No.: US 2002/0035232 A1
(43) Pub. Date: Mar. 21, 2002

(54) Mixed masked isocyanate composition and its use in powdered paint

(76) Inventor: Jean-Marie Bernard, Mornant (FR)

Correspondence Address: RHODIA INC., CN-7500, 259 Prospect Plains Road, CRANBURY, NJ, 08512, US

(21) Appl. No.: 09/973,257

(22) Filed: Oct. 9, 2001

Related U.S. Application Data
(63) Continuation of application No. 09/463,525, filed on Jan. 26, 2000.

(30) Foreign Application Priority Data
Jul. 30, 1997 (FR) 97/09723

(51) Int. Cl. C08G 18/80; C08L 75/00; C07C 271/06

(52) U.S. Cl. 528/45; 528/49; 252/182.2; 525/124; 525/424; 525/454; 560/24; 560/25; 560/26; 560/115; 560/157; 560/158; 560/330; 560/335; 560/336; 560/355; 560/358

(57) ABSTRACT

The invention concerns an isocyanate composition at least partially masked, characterized in that it is masked by at least two agents, one of which at least has a non-carbon carboxylic function. The invention is applicable to organic synthesis.

The present invention relates to a novel family of masked isocyanates. The invention relates more particularly to isocyanates masked using two masking agents, and to their use in coating techniques using powders.

For reasons associated with environmental protection and safety at work, efforts are increasingly being made to eliminate the use of solvents in coating techniques, and in particular in painting.

In this context, coating techniques using powders are being developed more and more.

Masked isocyanates are beginning to be used, but their use is limited by the fact that there are so few compounds which satisfy the chemical requirements of the powders.

A first difficulty lies in the problem of finding masked isocyanates or mixtures of masked isocyanates which remain in powdered form under the usual storage conditions, it being possible for these conditions to vary considerably from one place to another. This implies that these compounds should have a relatively high melting point and/or glass transition temperature (Tg).

The derivatives which are the subject of the present study do not always have a sharp melting point and thus, in this case, an apparent melting point is determined, either using a Koffler block or using a method of the so-called capillary type (for example the so-called “Büchi” melting point).

A glass transition temperature can be measured by differential thermal analysis (DTA) techniques.

It is also necessary for these compounds to have glass transition temperatures and melting points that are low enough for them to be able to react under the conditions in which the powders are used.

The compounds derived from crosslinking reactions should, in addition, not be harmful either to human or animal health or to the environment.

Consequently, one of the aims of the present invention is to provide a novel family of masked isocyanates which meets the constraints outlined above.

Another aim of the present invention is to provide compositions which can be used in powder coating and which contain masked isocyanates.

Another aim of the present invention is to provide a process for synthesizing isocyanates which meet the above constraints.

These aims and others which will become apparent hereinbelow are achieved by means of a composition whose isocyanate component is formed from at least partially masked isocyanate(s), said isocyanate component bearing at least one non-carbon-based carboxylic function masked with at least one masking agent.

The carboxylic function is advantageously in intimate mixture with the isocyanate component of the composition. To do this, it is even desirable for said carboxylic function to be grafted onto the composition by reaction of a precursor of said composition, of an agent bearing a carboxylic function and of a function which reacts with a free isocyanate function. In other words, it is desirable for the carboxylic function to be grafted onto the isocyanate system via an agent bearing both a function which is reactive (i.e. which contains a “labile” hydrogen) with the isocyanate function and a carboxylic function (COOH in salt form or, advantageously, in free form).

This agent bearing a carboxylic function and a function which is reactive (i.e. which contains a so-called labile hydrogen) with the isocyanate function is advantageously a masking agent. This means that it is released (octanol test) at a temperature of not more than 250° C., advantageously 200° C. (2 significant figures), preferably 180° C.

When the carboxylic functions are borne by agents which are not released under the firing conditions, in order to conserve the crosslinking power, it is desirable for the amount of acid borne by the agents which is not released to be not more than ½, advantageously ⅓ of the total amount of the isocyanate functions (which are free, masked and linked to a non-releaseable masking agent).

The masking agents most commonly used are those mentioned by M. Wicks in his article “blocked isocyanates” [Progress in Organic Coatings (1975), Vol. 3, p. 73].

In the context of the present invention, the preferred masking agents are those whose temperature for deblocking (or demasking) from the aliphatic isocyanates is at least equal to 90° C., advantageously 100° C. (two significant figures), preferably 110° C. (cf. below: octanol test).

It is desirable for the glass transition temperature (Tg) of the masked isocyanate, when the masking agent under consideration is used alone to mask the isocyanate targeted by the person skilled in the art, not to be very much below 0° C. (temperature at least equal to about 260° K, advantageously about 270° K, preferably about 280° K). This is because the carboxylic function, in general, hardly makes it possible to increase the glass transition temperature (Tg) by more than 30° C. This is particularly true for the preferred isocyanates (such as HDT) of the present invention, i.e. the isocyanates mainly obtained (i.e. representing at least a half) from monomers (for example HDI) which contain at least one, advantageously two, aliphatic functions which are neither secondary nor tertiary nor neopentyl.

In the present description, the term “about” is used to highlight the fact that the values following it correspond to mathematical round-ups, and in particular that when the figure(s) furthest to the right in a number is (are) zeros, these zeros are positional zeros rather than significant figures, except, of course, where otherwise mentioned.

The masking agents can be divided into three main groups:

those in which the labile hydrogen is borne by a chalcogen,

those in which the labile hydrogen is borne by a nitrogen,

those in which the labile hydrogen is borne by carbon.

Among those in which the labile hydrogen is borne by a chalcogen (preferably light chalcogens, i.e. sulfur and oxygen), the ones especially used are those in which the chalcogen is an oxygen; among the latter, mention may be made in particular of:

products containing an >N—OH linkage, such as, for example, oximes (═N—OH) or hydroxy imides ([—CO—]₂N—OH); and

phenols (in the broad sense), especially those in which the aromatic ring is electron-poor, such as hydroxypicolines and hydroxybenzoates (cf. for example EP-A 680 984 and WO 98/4608).

Mention may also be made of the compounds described in patent application EP-A 661 278.

Among those in which the labile hydrogen is formed by a nitrogen, mention may be made in particular of:

monosubstituted amides, and in particular lactams (the one usually used is caprolactam),

imides ([═CO—]₂N—H), especially cyclic amides such as succinimide,

unsaturated nitrogenous heterocycles, in particular those which are 5-membered (advantageously doubly unsaturated), preferably comprising at least two hetero atoms (preferably nitrogen); among the latter, mention may be made of diazoles (such as glyoxalines and pyrazoles), triazoles or even tetrazoles.

mention may also be made of the compounds described in patent application EP-A 661 278.

The masking agents in which the labile hydrogen is borne by carbon are essentially compounds of malonic nature, i.e. a radical RCH< bearing two electron-withdrawing groups (such as carbonyl [for example ketone, acid, ester or even acid salt], nitrile, Rf or [perfluoroalkyl]).

In order to avoid excessively weighting the masked molecules, it is desirable for the masking agents not bearing said non-carbon-based carboxylic function to contain not more than 10 carbon atoms, advantageously not more than 7 carbon atoms, preferably not more than 5.

It is easier and thus preferable for the masking group to bear the labile hydrogen which reacts with the isocyanate function via an oxygen or a nitrogen to give the —NH—CO—O— sequence (i.e. R—NCO+HO—→R—NH—CO—O—) or the sequence —NH—CO—N< (i.e. R—NCO+HN<→R—NH—CO—N<).

Thus, it is preferable for said isocyanate component to be a composition masked with at least two masking agents, at least one of which contains a non-carbon-based carboxylic function, i.e. the carboxylic function does not bear a carbon-based radical apart from the one which corresponds to the acid, and thus a non-carbon-based carboxylic function which does not comprise esters.

Advantageously, said non-carbon-based carboxylic function is chosen from acids and salts thereof (preferably inorganic salts). It is desirable for said non-carbon-based carboxylic function to be an acid function (—COOH). The free acid form is preferred.

Thus, it is possible to envisage several groups (preferably 2 for reasons of economy) for masking the isocyanate functions. This diversity can be achieved by mixing various masked compounds (in general masked with a single group) or preferably by co-reaction (successive, by reacting one and then the other masking agent, or simultaneous, by carrying out the masking action on the mixture of masking agents).

The presence of a carboxylic acid function (—COOH) and in particular of a carboxylic acid function directly grafted onto an aromatic ring, advantageously a benzene ring, makes it possible to increase the melting point of the masked isocyanate. However, in order to conserve the excellent mechanical properties of the system according to the present invention, it is preferable for the amount of acid function present (in equivalents) to be not more than about 9/10, advantageously not more than about ⅘, preferably not more than ⅔ of the masked isocyanate functions. The beneficial effects of the acid functions on the glass transition temperature (Tg) already become apparent above about 10%, but it is desirable to achieve a proportion of at least 20%. The melting point and the glass transition temperature (Tg) increase continually as the acid content increases, up to 100%. However, it is preferable for the content of masking agent bearing a non-carbon-based carboxylic function to be not more than 90%.

According to one embodiment of the present invention, these groups masking an isocyanate component may all be as defined above (cf. formula (I)) or alternatively only some of them may satisfy these definitions. In the latter case, it is preferable for those (i.e. the sum of those) which bear a carbonyl (ester or acid) function advantageously to correspond to formula (I) below and to correspond at least to about 10% (expressed as masked isocyanate function), advantageously about 20%, preferably to ⅓.

Thus, one of the most advantageous embodiments consists in using isocyanates which are at least partially masked with a compound bearing an acid function, advantageously with a compound of formula I in which Z is an acid function. In this case, it is advisable that the isocyanate be masked with a group other than the one bearing the acid function, and that the acid function of the masking system be between 90 and 10% (expressed as masked isocyanate function). The other masking agent(s) may be either masking agents that are known per se (which meet the constraints of release temperature or the masking temperature specified in the present description), or an ester corresponding to formula I. This latter term of the alternative is one of the preferred embodiments.

For the synthesis of the compounds according to the present invention, reference may be made to the general procedures, in particular those in patent application No. EP 0 680 984 A, which give good results for the (optionally partial) masking operation with a compound of formula I.

Curiously, the presence of a carboxylic group does not pose any problem for the masking operation. The masking functions react as if the carboxylic groups were not there. However, it is preferable to carry out the masking reaction at temperatures of not more than about 150° C., advantageously 130° C., preferably 110° C. (if a particularly low degree of free isocyanate is desired).

The presence of organic base catalyzes the masking reaction (see below) and generally does not exceed 10% (as masked isocyanate function equivalents), or even 5%.

It is preferable to carry out the reaction such that, at the end of the additions, the system is close to the isocyanate/masking agent stoichiometry ±10%, 5%, 2%.

As has already been mentioned according to the present invention, it is preferable for the melting point of the compound or of the mixture of compounds obtained to show an apparent melting point at least equal to 30° C., preferably 50° C.

It is also preferable for the glass transition temperature to be at least equal to 20° C., advantageously at least equal to 40° C.

It is preferable to select the compounds according to the present invention such that they react completely with a primary alcohol at 250° C. in less than half an hour.

It is considered that the reaction is complete if it proceeds to 90% or more.

As has been mentioned above, the isocyanates for which the invention is most advantageous are those in which the nitrogen atom is linked to an sp³-hybridized carbon, and more particularly aliphatic ones, and in particular polymethylene diisocyanates (for example TMDI, tetramethylene diisocyanate, and HMDI [hexamethylene diisocyanate=OCN—(CH₂)₆—NCO]) and the various condensation derivatives thereof (biuret, etc.) and di- and “trimerization” derivatives thereof, (in the field considered, the term “trimer” refers to mixtures derived from the formation of isocyanuric rings from three isocyanate functions; in fact, along with the true trimer, there are heavier products derived from trimerization).

According to the present invention, it is desirable and sometimes necessary for the percentage of residual free isocyanate function (expressed as equivalents) attached to a skeleton comprising an exocyclic polymethylene chain (cf. above) to be not more than 5%, advantageously not more than 3%, preferably not more than 1%. The highest melting points or glass transition temperatures are obtained with percentages not exceeding 0.5%. The contents of aromatic derivatives hydroxylated on the ring are also advantageously low, i.e. not more than 5%, advantageously not more than 3%, preferably not more than 1%.

On the other hand, up to a mass content of ⅓, the presence in said isocyanate of unmasked oligomer (especially trimer) or oligo-condensates of cycloaliphatic monomers such as IPDT or nBDT has a very favorable effect on the glass transition temperature (Tg) and does not in any way impede the production of a high-quality coating. Such compositions can be readily obtained by mixing the unmasked trimer in the molten masked compound.

According to the present invention, the isocyanate is advantageously masked with at least one masking group bearing at least one function derived from acidic functions and in particular acid and ester functions. The masking can be mixed and can involve several masking groups.

It is desirable that, in the structure of the isocyanate(s), the part of the skeleton which connects two isocyanate functions should contain at least one polymethylene sequence (CH₂)π in which π represents an integer from 2 to 10, advantageously from 4 to 8. This preference affects the mechanical performance. When there are several sequences, these sequences can be the same or different. In addition, it is desirable for at least one, preferably all, of these sequences to be freely rotating and thus exocyclic.

The degree of release is quantified by the octanol test (see below).

According to the present invention, the masked isocyanate, pure or mixed, is obtained from a polyisocyanate, i.e. a product-containing at least two isocyanate functions, advantageously more than two (possibilities of fractional values since it is generally a mixture of more or less condensed oligomers), which is itself usually derived from a precondensation or a prepolymerization of unitary diisocyanate (sometimes referred to in the present description as “monomer”).

In general, the average molecular mass of these prepolymers or of these precondensates is not more than 2000 (one significant figure), more commonly not more than 1000 (one significant figure, preferably two).

One notable exception to these values lies in precondensates between polyols with a molecular mass of greater than 2000 and less than 15,000. This relates to the number-average molecular mass, \overline{M}n, which ranges between 2000 and 15,000 g/mol. The molecular mass is determined by gel permeation chromatography (GPC). The gels used in the technique are two polystyrene gels (Ultrastyragel® at 10⁴ and 500 Å), THF as solvent and sulfur as standards. However, these prepolymers are not always the best.

Thus, among the polyisocyanates used for the invention, mention may be made of those of the biuret type and those in which the di- or trimerization reaction has led to four-, five- or six-membered rings. Among the six-membered rings, mention may be made of the isocyanuric rings derived from a homo- or hetero-trimerization of various diisocyanates alone, with other isocyanate(s) [mono-, di- or polyisocyanate(s)] or with carbon dioxide. In this case, a nitrogen of the isocyanuric ring is replaced with an oxygen. Oligomers containing isocyanuric rings are preferred.

The preferred polyisocyanates are those which contain at least one aliphatic isocyanate function. In other words, at least one masked isocyanate function according to the invention is connected to the skeleton via an sp³-type carbon advantageously bearing a hydrogen atom, preferably two. It is desirable for said sp³-type carbon itself to be borne by an sp³-type carbon and advantageously to contain one, preferably two, hydrogen atoms, so as to avoid the isocyanate function concerned being in a neopentyl position. In other words, it is recommended to choose as monomers (which, in general, bear two isocyanate functions) at least one compound which bears at least one aliphatic function which is neither secondary nor tertiary nor neopentyl.

In the case of the mixture obtained from several (in general two) types of monomer, it is preferable for the monomer(s) which satisfy the above conditions and/or (advantageously “and”) the condition regarding the presence of a polymethylene sequence (CH₂)π, to represent at least ⅓, advantageously ½, preferably ⅔ of the masked isocyanate functions. Thus, during the study according to the present invention, excellent results have been obtained with mixtures comprising ⅔ of HMDT (hexamethylene diisocyanate “trimer”) with IPDI or IPDT (IPDI “trimer”), the two being masked according to the invention (the nBDI, norbornane diisocyanate, and its trimer are similar).

Needless to say, the case in which all of the isocyanates are aliphatic and, what is more, satisfy the above criterion is preferred.

According to one particularly advantageous embodiment of the present invention, an agent corresponding to the general formula of those targeted in European patent application EP-A 661 278 can be used as masking agent bearing said non-carbon-based carboxylic function. The other agent(s) can also correspond to said general formula, as is the case for the para-hydroxybenzoic acid/alkyl para-hydroxybenzoate couple. The essence of EP-A 661 278 will be found below.

The agents leading to the characteristic masking group(s) of the invention are advantageously chosen from those which are derived from the condensation of an aromatic derivative which is hydroxylated on the ring and bears a function chosen from nitrile functions, and preferably carbonyl functions, with an isocyanate. It goes without saying that the condensation takes place on the phenol function.

Among the members of this family, those for which it is possible to determine an apparent melting point should be selected, this measurement being carried out at room temperature (20° C.). This melting point should be at least equal to 30° C. (one significant figure), advantageously at least equal to 50° C.

Among the compounds of formula (I), acids and salts thereof (alkali metal, alkaline-earth metal, ammonium and/or quaternary phosphonium salts) will be chosen as first masking agent, but the others can be chosen as second or third masking agent.

Ar(R)ₙ(Y—Z)ₘ(OH)ₚ (I)

in which Ar is an aromatic residue onto which are grafted n substituents R, m polar functions Z chosen from nitrites and carbonyl groups, and p hydroxyl functions.

The values of n, m and p are such that the sum n+m+p is not more than the number of substitutable members; advantageously, p is not more than 2 and is preferably equal to 1.

Advantageously, m is not more than two and is preferably equal to 1.

Advantageously n is not more than 3, it is preferably chosen from zero, 1 and 2, and is more preferably equal to zero.

R represents substituents that have no bearing on the masking reaction and generally correspond to hydrocarbon-based chains, usually alkyl chains in the etymological sense of the term, i.e. an alcohol whose hydroxyl function has been removed.

Two vicinal substituents R can be joined together to form a ring which can be aromatic, for example.

Z is advantageously chosen from groups containing a carbonyl function. Among these functions, mention should be made of alkoxycarbonyl functions (or, in other words, ester functions), the amide function, the ketone function with the preferential condition that there are no acidic hydrogens [in other words, the function advantageously does not bear hydrogen, or, if it does bear hydrogen, the corresponding pKa is at least equal to about 20 (one significant figure, preferably 2), more preferably at least equal to about 25] α to the carbonyl function (ester, ketone or amide]. Thus, the preferred amides (including lactam, or even urea) are advantageously substituted, preferably enough for there to be no hydrogen on the nitrogen of the amide function or such that there are no reactive hydrogens.

In which Y is chosen from divalent groups, advantageously —O—, —S—, NR′—, —CR′R″— with R′ and R″ chosen from hydrocarbon-based radicals, advantageously alkyl radicals, of 1 to 6 carbon atoms, advantageously of 1 to 4 carbon atoms, preferably methyl, more preferably hydrogen, and preferably Y represents a single bond [sic].

It is preferable for the polar function(s) Z (in general chosen from the nitrile function and/or carbonyl functions) not to be vicinal to the group Z, as, for example, in salicylic acid.

The aromatic residue Ar consists of one or more advantageously fused, hetero- or homocyclic rings. It is preferable for Ar not to contain more than two rings, and preferably not more than one ring.

The aromatic residue Ar can consist of one or more hetero- or homocyclic rings, usually homocyclic on account of their ease of access. However, the advantage of 6-membered heterocycles, which have a much lower release temperature than that of the corresponding homocycles, should be pointed out.

It is desirable for the total number of carbons in the aromatic derivative hydroxylated on the ring to be not more than 20, preferably not more than 10 (one significant figure).

This ring is advantageously 6-membered, the members consisting of carbon or nitrogen with the number of substituents required for the valency of these atoms.

Among the acids and derivatives, in particular esters, which give the most satisfactory results, mention should be made of acids grafted onto a benzene ring or onto pyridine rings. Thus, meta-hydroxybenzoic acids, and especially para-hydroxybenzoic acids, and derivatives thereof give good results.

According to one particularly advantageous variant of the present invention, the hydroxyarylcarboxylic acids, and in particular the hydroxybenzoic acids, can be combined with one (or more) of the usual masking agents, such as oxime, lactam, pyrazole or triazole, provided that they satisfy the glass transition temperature (Tg) and melting point constraints.

The combinations comprising at least one triazole, which is advantageously unsubstituted, and at least one agent bearing a carboxylic function, chosen in particular from hydroxyarylcarboxylic acids, are particularly advantageous.

Thus, it is desirable for said masking agent containing a carboxylic function to represent, in equivalents, at least 10% of all the masking agents, advantageously at least 20% of all the masking agents, preferably ⅓, but the effects are very marked when the proportion approaches or exceeds 50%.

However, when the carboxylic functions are borne by agents which are not released under the curing conditions, in order to conserve the crosslinking power, it is desirable for the amount of acid borne by agents which are not released to be not more than ½, advantageously ⅓ of the total amount of isocyanate functions (free, masked or linked to an unreleaseable masking agent).

In order to promote the solid form, said masking agent bearing a carboxylic function has, per masking agent, not more than 4, advantageously 2, freely rotating methyl or methylene groups (i.e. groups not engaged in a ring).

In order to reduce the firing (crosslinking) temperature, urethane-forming catalysts such as DBTDL (dibutyltin dilaurate) can be added to the formulation, these being added directly into the premix or as a stock mixture.

The polyols which can be used with the compounds according to the present invention are those known to those skilled in the art.

This is likewise the case for the additives.

The powdered paints which form the subject of the present invention can advantageously be manufactured by fusion-blending the compounds of the formulation. First, they are preblended in a blender and then melted, homogenized and dispersed in a single-screw or multi-screw extruder.

It is desirable for the mixing, blending and extrusion temperature not to be more than about 130° C., advantageously not more than about 110° C., preferably not more than 100° C. (3 significant figures). It is desirable for the extrusion temperature to be at least equal to about 60° C., advantageously at least equal to about 70° C., preferably at least equal to around 80-90° C. The extruded material obtained is left to cool, it is passed through a mill until a paint of the desired particle size is obtained (in general, d₉₀ is not more than about 200 micrometers, advantageously not more than 100 micrometers (two significant figures) and d₁₀ is at least equal to about 20 micrometers, advantageously at least equal to about 50 micrometers) for the purpose of applying it to metal supports such as steel, aluminum or other alloys, glass, plastic or wood.

The ratio between the polyol(s) and the isocyanate(s) is defined by the deblocking stoichiometry. The amount of isocyanates chosen is generally the amount stoichiometrically required to react with all of the free hydroxyls, with a tolerance of 20%, advantageously of 10%, preferably of 5%. Since it is preferable to have an excess of isocyanates, slightly unequal ranges are preferred. In other words, the amount of isocyanates to be added is advantageously at least equal to about 90% and not more than about 120% of the stoichiometric amount; preferably, it is at least equal to 95% and not more than about 110% of the stoichiometric amount; the most common, and thus most desirable, range being at least equal to 100% (three significant figures) and not more than 105% of the stoichiometric amount. When systems with a high proportion of free acid (for example at least ⅔ of the masked isocyanate functions, see above) are used, it may be envisaged to increase the ratio between isocyanate and hydroxyl function by about 10 to 30 points (%) relative to the above values.

The catalysts which can be used as catalysts for the masking reaction do not in any way harm the quality of the coatings. If formulations are used which favor a matt or satin coating, they accentuate the matt nature of the coating, in particular in the case of formulations whose matt nature is associated with carboxylic functions (see in particular PCT patent application WO 98/04608). When glossy formulations are used, they promote crosslinking.

Thus, organic bases in which the basic function is borne by a nitrogen or phosphorus atom, nitrogen being preferred, can be present in the composition. It is desirable for the basic atom not to bear any hydrogen. The basicity is at least equal to that of pyridine rings (for example pyridine itself, picolines or quinoline). The preferred bases are phosphines or, preferably, tertiary amines. The tertiary amines can contain from 3 up to about 50 carbon atoms per basic function (it is recalled that it is more practical for there to be only one per molecule). Heavy amines, especially fatty amines, have a favorable effect on the smooth aspect of a painted surface.

Amines also have a favorable effect on the mechanical properties, which indicates a catalysis of the crosslinking in the powder phase.

Admittedly, it is preferable for the organic bases to be, per se, relatively non-volatile (b.p. at least equal to 80° C., advantageously 100° C., preferably 200° C.), but, under the conditions for crosslinking powders, the observation of the low volatility is not very restricting and is, in particular, easily achieved provided that there are free acid functions in the binder in an amount at least equal to the amount of base (expressed in equivalents). If not, it is possible to envisage using heavier bases, i.e. bases whose molecular mass is at least equal to 100, advantageously 180, preferably fatty bases, i.e. bases whose molecular mass is greater than 250. The organic bases can be pure or a mixture. The amines can comprise other functions and in particular functions corresponding to amino acid functions and cyclic ether functions such as N-methylmorpholine, or not. These other functions are advantageously in a form which does not react with the isocyanate functions.

The amount of amine can be at least equal to about 1% (in equivalents), advantageously 2%, preferably 3% of the masked isocyanate functions in the powder. The amines can be used alone or in combination with the other components of the powder paints.

The powder obtained can be applied with an electrostatic gun or by means of a fluid bed. The preferred application of the present invention is that carried out with a Corona effect and Corona charge electrostatic gun or by friction (triboelectric).

The substrate onto which the paint is applied, mainly steel or aluminum, may or may not be preheated before the application. Once applied, the powder is melted and cured in an oven for 10 minutes to 2 hours, at a temperature of between 140 and 220° C. depending on whether or not the system is catalyzed, in general for 10 to 30 minutes at a temperature ranging from 180° C. to 220° C.

Given the account hereinabove, a person skilled in the art will adapt the firing recalling that increasing the firing temperature allows the time to be reduced, and vice versa.

The isocyanate compositions and components according to the present invention can be used in powder form. They can also be used in dissolved form. They are of specific value for use in the form of a suspension, especially when the content of free isocyanate is low (free isocyanate(s)/total isocyanate ratio (in equivalents) advantageously not more than 5%, preferably 2%).

The non-limiting examples below illustrate the invention.

In all the examples, HDT or HMDT means hexamethylene diisocyanate trimer known under the trade name Tolonate® HDT and IPDT means isophorone diisocyanate trimer.

Octanol Test

Definitions

| | |
|---|---|
| “Release” (or | This is the lowest temperature at |
| “deblocking”) | which the agent masking the masked |
| temperature: | isocyanate is displaced to an |
| | extent of 9/10 (mathematically |
| | rounded-up) by a primary |
| | monoalcohol (the primary alcohol |
| | is generally octanol). |
| Shelf life: | In order to ensure a good shelf |
| | life, it is preferable to choose |
| | masked isocyanate functions whose |
| | octanol test shows a “release” at |
| | 80° C., advantageously at 90° C., of |
| | not more than 90%. |
| Reaction progress: | The reaction is considered to be |
| | complete if it proceeds to more |
| | than 90%. |

Procedure

About 5 mmol of protected masked NCO equivalent to be evaluated are loaded into a Schott-type tube with magnetic stirring.

2.5 to 3 ml of 1,2-dichlorobenzene (solvent) and the equivalent of 1-octanol (5 mmol, i.e. 0.61 g, optionally with the catalyst to be tested with the masking group) are added.

The reaction medium is then brought to the test temperature. It is then heated for 6 h at the test temperature so as to deblock the isocyanate functions and thus make them reactive. Once the reaction is complete, the solvent is removed by distillation under vacuum and the residue is analyzed by NMR, mass spectrum and infrared.

From these data, the percentage of masked isocyanate function condensed with the 1-octanol is evaluated.

When the boiling point of one of the constituents is higher than the temperature which it is desired to test, the process is then performed at the corresponding autogenous pressure.

### EXAMPLE 1

### Of the Synthesis of HDT Masked With a Mixture (80/20 mol per mole) of methyl para-hydroxybenzoate and para-hydroxybenzoic Acid

136.2 g of Tolonate® HDT product, whose content of isocyanate (NCO) functions is 0.521 mol per 100 g of HDT, are introduced into a 500 ml thermostatically-controlled reactor fitted with a mechanical stirrer. 86.4 g of methyl para-hydroxybenzoate and 19.6 g of para-hydroxybenzoic acid are successively added. The temperature of the reaction medium [lacuna] brought to 86° C. and 3.7 g of triethylamine are then added. The temperature of the reaction medium is then brought to 100° C. After 2 h 45 min at 100° C., the reaction medium is cooled and then ground to give a powder. The content of free isocyanate functions is 1.05% and the Tg of the product is about 30° C.

### EXAMPLE 2

### Of the Synthesis of HDT Masked With a Mixture (65/35 mol per mole) of methyl para-hydroxybenzoate and para-hydroxybenzoic Acid

200 g of Tolonateo HDT product whose content of isocyanate (NCO) functions is 0.521 mol per 100 g of HDT are introduced into a 500 ml thermostatically-controlled reactor fitted with a mechanical stirrer. 113.3 g of methyl para-hydroxybenzoate and 50.4 g of para-hydroxybenzoic acid are successively added. The temperature of the reaction medium is brought to 26° C. and 3 g of triethylamine are then added. The temperature of the reaction medium is then brought to 93° C. After 30 minutes at 93° C., the temperature of the reaction medium is brought to 130° C. The molten mass is removed, cooled and then ground to give 344 g of powder. The content of free isocyanate functions relative to the HDT is 1.3% and the Tg of the product is about 32° C. Infrared analysis in a KBr disk indicates the presence of peaks for the acid function at 2500 cm⁻¹, a very low content, or even an absence, of peaks corresponding to the acid functions (1650 cm⁻¹ and 1550 cm⁻¹) and the presence of carbamate functions.

The content of potential isocyanate functions is 12.03%.

### EXAMPLE 3

### Of the Synthesis of HDT Masked With a Mixture (50/50 mol per mole) of methyl para-hydroxybenzoate and para-hydroxybenzoic Acid

The same method as described in Example 2 is applied. 342 g of white powder are thus recovered.

The content of free NCO, expressed as unmasked HDT, is 0.7%, i.e. 0.175% by weight of residual NCO functions. The Tg of the product is about 35° C.

The content of potential NCO is 11.95%.

Infrared analysis shows the characteristic peaks of the expected product.

### EXAMPLE 4

### Of the Synthesis of a Mixture (85/15% by Weight) of (HDT/IPDT) Masked With a Mixture (65/35 mol per mole) of methyl para-hydroxybenzoate and para-hydroxybenzoic Acid

The same procedure as in Example 2 is used, except that the mixture of the two polyisocyanates HDT and IPDT (119 g and 21 g) is introduced instead of the HDT, the amounts of masking agent are adjusted and the removal takes place at 160° C. After removal and cooling, the product is ground.

The Tg of the product is about 35° C. and the content of free isocyanate functions is 0.2% by weight, i.e. 0.82% of unmasked HDT.

The infrared spectrum shows the peaks characteristic of the expected product.

### EXAMPLE 5

### of the synthesis of HDT masked with a mixture (94/6 mol per mole) of methyl para-hydroxybenzoate and calcium salt of para-hydroxybenzoic Acid

141.25 g of Tolonate® HDT product whose NCO content is 0.521 mol per 100 g of HDT are introduced into a 500 ml thermostatically-controlled reactor fitted with a mechanical stirrer. 111.9 g of methyl para-hydroxybenzoate, 6.07 g of para-hydroxybenzoic acid and 4.4 g of calcium carbonate are successively added. The temperature of the reaction medium is brought to 120° C. and maintained for 45 minutes. The reaction mixture is cooled to 80° C. and 1.3 g of triethylamine are then added. The temperature of the reaction medium is then brought to 95° C. The reaction medium is removed and ground at room temperature to give a powder. The content of free NOC [sic] is 0.69% and the Tg of the product is about 26° C.

### EXAMPLES 6 TO 19

### Of the Synthesis of HDT Masked With a Mixture of Two Masking Agents, One of Which Bears a Carboxylic Function

The procedure is, with the necessary changes (masking agents and percentage thereof), that of Example 2 when the isocyanate is HDT alone and that of Example 3 when the isocyanate is an HDT/IPDT mixture.

| | Nature and | | | | |
|---|---|---|---|---|---|
| | percentage by mass | | Masking agents as a | | |
| | of polyisocyanates | | mol % relative to | | Tg |
| | polyiso- | polyiso- | the NCO functions | | in |
| Comp. | cyanate 1 | cyanate | Agent 1 | Agent 2 | ° C. |
| Examples | HDT/100 | | triazole/100 | | <0 |
| 7 | HDT/100 | | triazole/90 | p-hydroxy- | 19 |
| | | | | benzoic | |
| | | | | acid/10 | |
| 8 | HDT/100 | | triazole/80 | p-hydroxy- | 27 |
| | | | | benzoic | |
| | | | | acid/20 | |
| 9 | HDT/100 | | triazole/70 | p-hydroxy- | 29 |
| | | | | benzoic | |
| | | | | acid/30 | |
| 10 | HDT/100 | | triazole/60 | p-hydroxy- | 30 |
| | | | | benzoic | |
| | | | | acid/40 | |
| 11 | HDT/100 | | triazole/50 | p-hydroxy- | 38 |
| | | | | benzoic | |
| | | | | acid/50 | |
| 12 | HDT/85 | IPDT/15 | triazole/85 | p-hydroxy- | 23 |
| | | | | benzoic | |
| | | | | acid/15 | |
| 13 | HDT/85 | IPDT/15 | triazole/70 | p-hydroxy- | 34 |
| | | | | benzoic | |
| | | | | acid/30 | |
| 14 | HDT/70 | IPDT/30 | triazole/85 | p-hydroxy- | 38 |
| | | | | benzoic | |
| | | | | acid/15 | |
| 15 | HDT/70 | IPDT/30 | triazole/70 | p-hydroxy- | 35 |
| | | | | benzoic | |
| | | | | acid/30 | |
| 16 | HDT/100 | | triazole/90 | para-amino- | 15-20 |
| | | | | benzoic | |
| | | | | acid/10 | |
| 17 | HDT/100 | | triazole/80 | para-amino- | 26 |
| | | | | benzoic | |
| | | | | acid/20 | |
| 18 | HDT/100 | | triazole/90 | N salt | 15-20 |
| | | | | (Nylon | |
| | | | | salt)*/10 | |
| 19 | HDT/100 | | triazole/65 | 6-amino- | 23 |
| | | | | caproic | |
| | | | | acid/35 | |

*the Nylon salt is the salt formed from the addition of hexamethylenediamine to adipic acid (mol/mol)

### EXAMPLE 20

### Qualitative Tests

The syntheses were carried out (according to procedure 2) for the following couples which show an increase in the glass transition temperature (Tg) relative to the compound containing no masking agent bearing a carboxylic function.

| | | carrier of non- |
|---|---|---|
| isocyanate | masking | carbon-based |
| used | agent | carboxylic function |
| HDT/100 | methyl p- | para-hydroxy- |
| | hydroxy- | phenylacetic |
| | benzoate/85 | acid/15 |
| HDT/100 | methyl p- | cholesteric |
| | hydroxy- | acid/15 |
| | benzoate/85 | |
| HDT/100 | methyl p- | hydroxyphthalic |
| | hydroxy- | acid/15 |
| | benzoate/85 | |
| HDT/100 | methyl p- | para-hydroxy- |
| | hydroxy- | benzamide |
| | benzoate/85 | acid [sic]/15 |

1. Isocyanate composition which is at least partially masked, characterized in that it comprises at least one non-carbon-based carboxylic function and is masked with at least one masking agent.

2. Composition according to claim 1, characterized in that said carboxylic function is grafted onto the composition by reaction of a precursor of said composition, of an agent bearing a carboxylic function and of a function which reacts with a free isocyanate function.

3. Composition according to claim 2, characterized in that said agent bearing a carboxylic function and a function which reacts with a free isocyanate function is a masking agent bearing a carboxylic function.

4. Composition according to claims 1 to 3, characterized in that said composition is a composition masked with at least two masking agents, at least one of which contains a non-carbon-based carboxylic function.

5. Composition according to claims 1 to 4, characterized in that the ratio (in equivalents) between the carboxylic functions, on the one hand, and the isocyanate functions (which are masked, free and which have reacted with any agent bearing carboxylic functions), on the other hand, is at least equal to 5%, advantageously 10%, preferably 20%.

6. Composition according to claims 1 to 5, characterized in that the ratio (in equivalents) between the carboxylic functions, on the one hand, and the isocyanate functions (which are masked, free and which have reacted with any agent bearing carboxylic functions), on the other hand, is not more than about 9/10, advantageously about ⅘, preferably ⅔, more preferably one third.

7. Composition according to claims 1 to 6, characterized in that when the carboxylic functions are borne by agents which are not released under the firing conditions, in order to conserve the crosslinking power, it is desirable for the amount of acid borne by agents which is not released to be not more than ½, advantageously ⅓ of the total amount of the isocyanate functions (which are free, masked and linked to an unreleaseable masking agent).

8. Composition according to claims 1 to 7, characterized in that said non-carbon-based carboxylic function is chosen from acids and salts thereof.

9. Composition according to claims 1 to 8, characterized in that said non-carbon-based carboxylic function is an acid function (—COOH).

10. Composition according to claims 1 to 9, characterized in that said masking agent containing a carboxylic function represents, in equivalents, at least 10% of all of the masking agents.

11. Composition according to claims 1 to 10, characterized in that said masking agent containing a carboxylic function represents, in equivalents, at least 20% of all of the masking agents.

12. Composition according to claims 1 to 11, characterized in that said masking agent containing a carboxylic function contains, per masking agent, not more than 4, advantageously 2, freely rotating methyl or methylene groups.

13. Composition according to claims 1 to 12, characterized in that said masking agent containing a carboxylic function corresponds to the formula:
Ar(R)ₙ(Y—Z)ₘ(OH)ₚ (I)
in which Ar is an aromatic residue to which are grafted n substituents R, m polar functions Z chosen from the carboxylic acid function and salts thereof, and p hydroxyl functions; the values of n, m and p are such that the sum n+m+p is not more than the number of substitutable members, advantageously p is not more than 2 and it is preferably equal to 1.

14. Composition according to claims 1 to 13, characterized in that said carboxylic function is directly linked to an aromatic ring.

15. Composition according to claims 1 to 14, characterized in that the masking agent not bearing a carboxylic function is chosen from masking agents which are known per se.

16. Composition according to claims 1 to 15, characterized in that the masking agent not bearing a carboxylic function is chosen from oximes, lactams, pyrazoles and triazoles.

17. Composition according to claims 1 to 16, characterized in that said isocyanate composition is formed from an oligo-condensate or an oligomer containing at least one masked aliphatic isocyanate function.

18. Composition according to claim 17, characterized in that said masked aliphatic function is connected to the skeleton via an Sp³-type carbon advantageously bearing a hydrogen atom, preferably two.

19. Composition according to claim 18, characterized in that said aliphatic function is neither secondary nor tertiary nor neopentyl.

20. Composition according to claims 1 to 19, characterized in that said isocyanate composition is obtained from an at least partial masking of a mixture obtained by oligomerization or oligocondensation starting with several monomers, at least one of which contains at least one, advantageously two, aliphatic function(s) which is(are) neither secondary nor tertiary nor neopentyl.

21. Composition according to claim 20, characterized in that the unit(s) obtained from monomer(s) which contain(s) at least one, advantageously two, aliphatic function(s) which is(are) neither secondary nor tertiary nor neopentyl bear(s) at least ⅓, advantageously ½ and preferably ⅔, masked isocyanate functions.

22. Composition according to claims 1 to 21, characterized in that said isocyanate composition is obtained from an at least partial masking of a mixture obtained by oligomerization or oligocondensation starting with several monomers, at least one of which contains a polymethylene chain, the unit(s) obtained from monomer(s) which contain(s) at least one polymethylene chain bearing at least ⅓, advantageously ½ and preferably ⅔, masked isocyanate functions.

23. Composition according to claims 1 to 22, characterized in that the masking agent not bearing a carboxylic function is chosen from triazoles.

24. Composition according to claims 1 to 23, characterized in that it also comprises an organic base, advantageously a tertiary amine.

25. Use, in a powder paint formulation, of the composition according to claims 1 to 24.

26. Coating, characterized in that it can be obtained according to claim 25.
